(19) United States
(12) Patent Application Publication
(10) Pub. No.: US 2022/0378400 A1
(43) Pub. Date: Dec. 1, 2022

(54) COMBINED ULTRASOUND AND ENDOSCOPY

(71) Applicant: Suzhou AcuVu Medecal Technology Co., Ltd., Suzhou, Jiangsu (CN)

(72) Inventor: Zengpin YU, Los Altos, CA (US)

(21) Appl. No.: 17/774,656

(22) PCT Filed: Jun. 11, 2020
(86) PCT No.: PCT/IB2020/000470

§ 371 Date: May 5, 2022

Related U.S. Application Data
(60) Provisional application No. 62/933,216, filed on Nov. 8, 2019.
(60) Provisional application No. 62/630,718, filed on Feb. 14, 2018.

(51) Int. Cl. A61B 8/00; A61B 8/12; A61B 1/00; A61B 1/05; A61B 1/307; A61B 1/005; A61B 8/08

(52) U.S. Cl. CPC A61B 8/4416; A61B 8/12; A61B 8/445; A61B 8/463; A61B 1/00045; A61B 1/05; A61B 1/307; A61B 8/54; A61B 1/00009; A61B 1/0052; A61B 1/00103; A61B 1/0057; A61B 8/5215; A61B 8/4455; A61B 1/00147; A61B 1/0005

(57) ABSTRACT

A combined ultrasonic and endoscopy system includes a cannula with a distal tip configured for insertion into an internal organ or other internal body structure. The distal tip is configured to include both an ultrasound probe head and a camera module. The ultrasonic and direct vision endoscopy images can be simultaneously displayed to a user on a display monitor. The ultrasound probe head can be rotated and steered to scan any location in the human organ cavity. The ultrasound probe can be re-usable or single use. The endoscopy system can be configured with a handheld portion that includes a re-usable handle portion and a single use portion that is configured to be disposed of following a single use. The system can also be configured using a conventional re-usable endoscope with working channels and an endoscopy processing tower system.

### REFERENCE TO RELATED APPLICATION

This application is: (a) a National Stage of International Patent Application PCT/IB2020/000470 filed Jun. 11, 2020 and claiming priority to U.S. Provisional Application No. 62/933,216 filed Nov. 8, 2019, and (b) a continuation-in-part of each of U.S. patent application Ser. No. 16/268,819 and U.S. patent application Ser. No. 16/268,909 both filed Feb. 6, 2019, each of which claims priority to U.S. Provisional Patent Application Ser. No. 62/630,718, filed on Feb. 14, 2018.

This application incorporates by reference and claims the benefit of the filing date of each of the above-identified patent applications, as well as of the applications that they incorporate by reference, directly or indirectly, and the benefit of which they claim, including U.S. provisional applications, U.S. non-provisional applications, and International Applications.

### FIELD

This patent specification generally relates to a medical device for use in tissue and organ examinations. More particularly, some embodiments relate to combined ultrasound and endoscopy systems for examining internal organs and other internal body structures.

### BACKGROUND

Ultrasound are sound waves with frequencies which are higher than those audible to humans (>20,000 Hz). Ultrasonic images, also known as sonograms, are made by sending pulses of ultrasound into tissue using a probe. The ultrasound pulses echo off tissues with different reflection properties and are recorded and displayed as an image. Medial ultrasound is conventionally used for creating images of internal organs as well as other body structures such as tendons, muscles, joints, and blood vessels. Although medical ultrasound commonly uses transducers designed to be used externally, such as through the lower abdominal wall in the case of gynecologic ultrasonography, occasionally the ultrasonic transducer is configured for insertion into an internal organ or other structure. One such example is a sonohysterogram procedure which is use for imaging of the uterus. The procedure includes inserting fluid and an ultrasound probe into the uterus and can provide sonographic images of uterine structures. Although the sonohysterogram procedure may be preceded by an endoscopy procedure to obtain direct vision images of the uterine walls, conventionally a sonohysterogram procedure is performed “blind” or without any live visual aid during the insertion of the ultrasound probe.

### SUMMARY

According to some embodiments, an integrated visual and ultrasound device comprises: an ergonomic handle configured for grasping by hand and having proximal and distal portions; a cannula extending distally from the distal portion of the handle and having a distal portion extending along a longitudinal axis; a distally facing camera secured at the distal portion of the cannula and having a camera field of view (FOV) encompassing a selected solid angle and a camera direction of view (DOV) that is angled relative to said axis; an ultrasound probe positioned at the distal portion of the cannula for both rotation about said axis relative to the distal portion of the cannula and for tilting relative to said axis; a probe steering mechanism mounted at the proximal end of the handle and operatively coupled with the ultrasound probe to selectively tilt the ultrasound probe relative to said axis over a selected angular range; and a probe rotation mechanism mounted at the proximal end of the handle and operatively coupled with the ultrasound probe to selectively rotate the ultrasound probe about said axis relative to the cannula.

According to some embodiments, said integrated visual and ultrasound device can further include one or more of the following features: the cannula can comprise at least one lumen and can further include a shaft connecting the probe steering mechanism and the ultrasound probe, wherein said shaft is removably received in said lumen; a belt that is in said shaft can be coupled to and driven by said probe rotation mechanism and gearing can be secured to said ultrasound probe and driven by said belt to selectively rotate the ultrasound probe about said axis; the probe rotation mechanism can be configured to rotate the ultrasound probe through at least 180 degrees; the ultrasound probe can be secured to a rotation plate that rotates about a pivot axis transverse to said longitudinal axis, and a bar inside said shaft can couple said probe steering mechanism to said rotation plate and respond to rotation of the steering mechanism to pivot the rotation plate and thus the ultrasound probe relative to said longitudinal axis; said steering mechanism can be configured to tilt said ultrasound probe in two opposite directions relative to said longitudinal axis through an angle up to 180 degrees in at least one of said directions; said steering mechanism can be configured to tilt said ultrasound probe through different angular ranges in said two opposite directions relative to said longitudinal axis; said handle can comprise (i) a multiple-use portion and image processing electronics therein coupled to said camera and said ultrasound probe and (ii) a single-use portion removably secured to the multiple-use portion and housing said rotation mechanism and steering mechanism; said cannula can be flexible to bend when inserted into a patient's bladder or ureter; an ultrasound image processor can be operatively coupled with said ultrasound probe and an ultrasound image display can be configured to display ultrasound images provided by said ultrasound probe and processed by said ultrasound processor, and a camera image processor and a camera image display can be configured to display images provided by said camera and processed by said camera image processor; said ultrasound image display and camera image display can be configured to concurrently display said ultrasound and camera images on a single screen; the ultrasound and visual aspects can be integrated by the ultrasound probe being inserted through a working channel formed within the cannula; said cannula can be configured such that at least a portion thereof has a stiffness property selected from a group consisting of: rigid, semi-rigid and flexible; the DOV can be within a range of 0 to 30 degrees; a cannula rotation mechanism can be positioned at the proximal portion of the handle and operatively coupled with the cannula to selectively rotate the cannula and thus the camera about said axis relative to the handle; said probe rotation mechanism can be a probe rotation wheel; a rotation sensor can be operatively coupled with the probe rotation mechanism and configured to provide an electronic signal indicative of rotation of the ultrasound probe about said axis; a processing system can be configured to process ultrasound images from said ultrasound probe and automatically generate therefrom three-dimensional ultrasound images based in part on said electronic signals from said rotation sensor; and said probe steering mechanism can be a probe steering wheel.

According for some embodiments, a medical device comprises: an elongated shaft having a distal portion extending along a longitudinal axis and a proximal portion, wherein the shaft is shaped and dimensioned for insertion into a sheath or a working channel of an endoscope cannula configured for insertion into a patient; an ultrasound probe that is at the distal portion of the shaft and is configured to protrude from a distal end of the sheath or cannula and to provide ultrasound images; a housing secured to the proximal portion of the shaft; a probe rotation mechanism mounted on or in the housing and operatively coupled with shaft to rotate the shaft and thus the ultrasound probe about said axis over a selected rotation angular range; and a probe steering mechanism mounted on or in the housing and operatively coupled with the ultrasound probe to tilt the ultrasound probe relative to said axis over a selected tilting angle range.

The medical device can further include one or more of the following features: a rotation sensor operatively coupled with the probe rotation mechanism and configured to provide an electronic signal indicative of rotation of the ultrasound probe about said axis; and said shaft can be shaped and dimensioned for insertion into a working channel of an endoscope that has a camera at a distal end thereof, wherein the ultrasound probe is configured to protrude distally from said camera when the shaft in inserted into said working channel.

According to some embodiments, a method comprises: providing an integrated camera and ultrasound imaging device comprising an elongated cannula having a distal portion extending along a longitudinal axis; inserting the cannula into an object; operating a camera mounted at a distal portion of the cannula to provide camera images of an interior portion of the object taken with a direction of view of the camera that is angled relative to said axis; operating an ultrasound probe also mounted at the distal portion of the cannula and protruding distally from said camera to provide ultrasound images of an interior portion of the object; selectively rotating the cannula and the camera about said axis through a selected rotation angle to view interior portions of the object from different directions, under manual control exerted at a proximal portion of the imaging device; selectively rotating the ultrasound probe about said axis and selectively tilting the ultrasound probe relative to said axis to obtain ultrasound images of selected portions of the object taken from different directions in a solid angle greater than 180 degrees, under manual control exerted a proximal portion of said imaging device; wherein operating the camera comprises including at least a partial image of the ultrasound probe in at least some of the images provided by the camera; processing the camera and ultrasound images; and displaying resulting processed camera images and ultrasound images.

According to some embodiments, the method can further comprise one or more of the following: sensing rotation of the ultrasound probe and controlling the display of ultrasound images as a function of the sensed rotation of the ultrasound probe; providing a handle configured for grasping by hand, wherein said cannula is secured to said handle; providing a handle comprising a single-use handle portion to which said device is secured and a multiple-use handle portion releasably secured to the single-use portion and including electronics for processing the ultrasonic images from the ultrasound probe; selectively withdrawing the ultrasound probe from the cannula while the cannula remains inserted in the object and inserting a surgical instrument in a cannula lumen vacated by the withdrawn ultrasound probe; selectively bending the cannula; said rotating the cannula is under manual control by rotating said integrated camera and ultrasound imaging device; said selectively rotating the cannula is under manual control by rotating a cannula rotation mechanism operatively coupled with the cannula to selectively rotate the cannula and thus the camera; and performing a surgical procedure on the object.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which principles of the invention are utilized, and the accompanying drawings of which:

FIGS. 1A-1C are schematic diagrams showing examples of combined ultrasound and endoscopy systems (CUES), according to some embodiments;

FIGS. 2A, 2B, 2C and 2D are right, left, top and front views of the handheld portion of a combined ultrasound and endoscopy system (CUES), according to some embodiments;

FIGS. 3A and 3B are perspective and top views of the distal end of a handheld portion of a combined ultrasound and endoscopy system (CUES), according to some embodiments;

FIGS. 4A and 4B are partial perspective views of a cannula forming part of a combined ultrasound and endoscopy system (CUES), according to some embodiments;

FIGS. 5A, 5B and 5C are perspective views showing further details of mechanisms for steering and rotation of an ultrasonic probe head that forms part of a combined ultrasound and endoscopy system (CUES), according to some embodiments;

FIG. 6 is a perspective view illustrating further detail of the distal tip of a combined ultrasound and endoscopy system (CUES), according to some embodiments;

FIG. 7 is a perspective view of a flexible handheld portion forming part of a combined ultrasound and endoscopy system (CUES), according to some embodiments;

FIGS. 8A and 8B are diagrams showing further detail of separable handheld portion of a combined ultrasound and endoscopy system (CUES), according to some embodiments;

FIGS. 9A-9C are diagrams illustrating further detail of the distal tip and cannula of a combined ultrasound and endoscopy probe, according to some embodiments;

FIGS. 10A-10C are diagrams illustrating further detail of the distal tip and cannula of a combined ultrasound and endoscopy probe, according to some embodiments; and

FIGS. 11A and 11B are schematic diagrams showing further examples of combined ultrasound and endoscopy systems (CUES), according to some embodiments.

### DETAILED DESCRIPTION

A detailed description of examples of preferred embodiments is provided below. While several embodiments are described, it should be understood that the new subject matter described in this patent specification is not limited to any one embodiment or combination of embodiments described herein, but instead encompasses numerous alternatives, modifications, and equivalents. In addition, while numerous specific details are set forth in the following description in order to provide a thorough understanding, some embodiments can be practiced without some or all of these details. Moreover, for the purpose of clarity, certain technical material that is known in the related art has not been described in detail in order to avoid unnecessarily obscuring the new subject matter described herein. It should be clear that individual features of one or several of the specific embodiments described herein can be used in combination with features of other described embodiments or with other features. Further, like reference numbers and designations in the various drawings indicate like elements.

As used herein a processor encompasses one or more processors, for example a single processor, or a plurality of processors of a distributed processing system for example. A controller or processor as described herein generally comprises a tangible medium to store instructions to implement steps of a process, and the processor may comprise one or more of a central processing unit, programmable array logic, gate array logic, or a field programmable gate array, for example.

As used herein, the terms distal and proximal refer to locations referenced from the apparatus and can be opposite of anatomical references. For example, a distal location of a probe may correspond to a proximal location of an elongate member of the patient, and a proximal location of the probe may correspond to a distal location of the elongate member of the patient.

While some exemplary embodiments are directed at cystoscopes and/or hysteroscopes, one of skill in the art will appreciate that this is not intended to be limiting, and the devices described herein may be used for other therapeutic or diagnostic procedures and in other anatomical regions of a patient's body.

According to various embodiments, a device includes a probing portion for direct insertion into a body cavity. The probing portion is brought into proximity to the tissue and/or area that is to be examined. As used herein, a probe encompasses an object inserted into a subject such as a patient.

According to some embodiments, an endoscopy ultrasound hysterography and cysterograph system (EUHCS) is described. According to some embodiments the system can be more generally described as combined ultrasound and endoscopy system (CUES). The EUHCS and CUES are medical devices that allow a doctor to obtain video images and organ information inside of uterus, bladder or other organs. According to some embodiments, the endoscopy images and ultrasound images are displayed at real time simultaneously on a monitor or two separate monitors allowing a doctor to see both the surface and inside tissue of organs and can be transmitted electronically to other devices such as work stations that can be at remote locations and/or PACS (Picture Archiving and Communication Systems).

In the case of gynecology clinical applications, according to some embodiments, applications for the EUHCS include: (1) early diagnosis of endometrial cancer; (2) providing endometrial cancer stage information by the depth, area and scope of cancerous infiltrating myometrium; (3) monitoring of hysteroscopic surgery to improve surgical safety and accuracy and success rate; and (4) detecting, diagnosing, and determining the stage of ovarian and/or fallopian tube cancers.

In the case of providing combined ultrasound and endoscopy images of the uterus, the devices and methods of this patent specification can account for the shape of uterus. The cervical canal and part of uterus cavity are cylindrical in shape. The lower surface of the upper uterine fundus is horizontally oriented with respect to the cervical canal and lower uterine cavity. According to some embodiments, the hysteroscope and ultrasound probe are configured to image in both vertical and horizontal directions.

Similarly, the shape of the bladder can be accounted for when providing combined ultrasound and endoscopy images of the bladder. The bladder is in a spherical shape. According to some embodiments, a flexible cystoscope and ultrasound catheter probe can be used to image the entire bladder or at least a desired portion thereof.

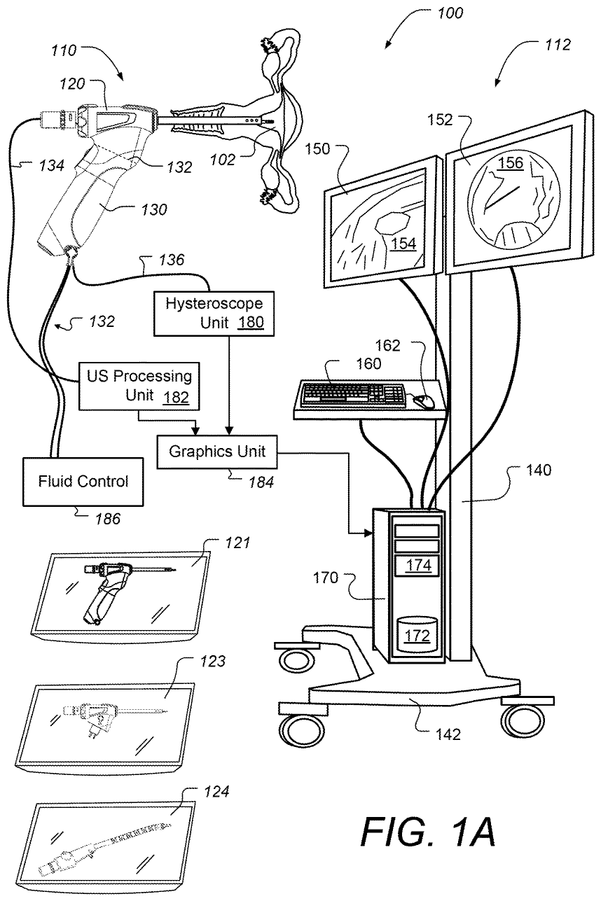

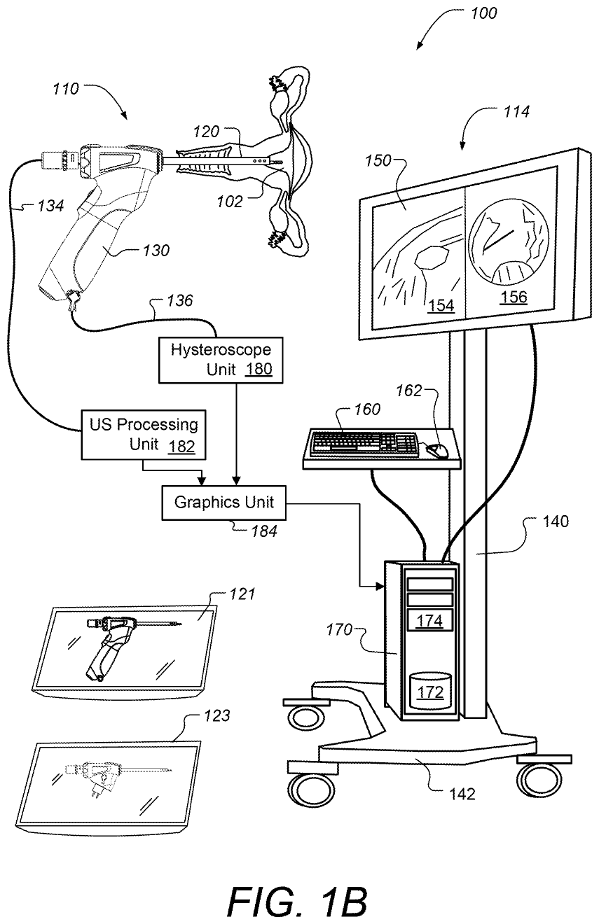

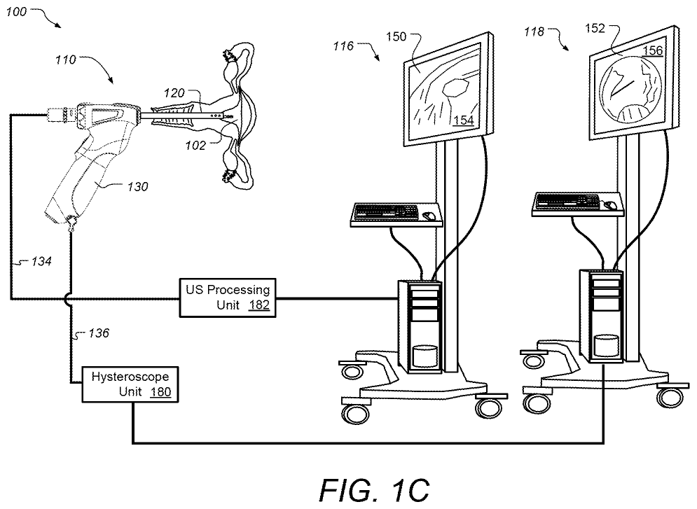

FIGS. 1A-1C are schematic diagrams showing examples of combined ultrasound and endoscopy systems (CUES), according to some embodiments. In FIG. 1A, the system 100 includes a handheld portion 110 and tower system 112 which are interconnected via cables 134 and 136 and processing units 180, 182 and 184. According to some embodiments, the handheld portion 110 is configured as a single-use unit and is disposable following a single use. According to some other embodiments, the handheld portion 110 is configured to separate into an upper single use portion 120 and a lower multiple-use handle portion 130. In such cases the single-use portion 120 is detachable from the handle portion 130, for example along dashed line 132, such that the handle portion 130 is configured to be used many times. According to some embodiments, different types of versions of the single-use portions can be made available for mating with the same multiple-use portion. In the example shown in FIG. 1, three versions of single-use portions are shown in sterile packages or pouches 121, 122 and 123. In pouch 121 an entire handheld portion 110 is being supplied. In pouch 123, a separable upper single use portion is being supplied for mating with the same multiple-use portion shipped in pouch 121 or with a like multiple-use portion, and in pouch 124, a flexible cannula version is being supplied. As will be described in further detail, infra, all of the devices can include a camera module, LED illumination, and ultrasonic transducer modules on their distal tips as well as one or more internal lumens for carrying fluid. The tower system 112 includes column 140 mounted to a wheeled base 142. The tower system 112 includes two displays 150 and 152, keyboard 160, a mouse 162 and processing system 170. The processing system 170 can functionally include ultrasound image processing unit 182, endoscopy optical image processing unit 180 (which is labeled Hysteroscope Unit in the Figures) and graphics unit 184 to perform image display and management processing. According to some embodiments, display 150 is configured to display ultrasound images 154 from the ultrasound transducer module on the distal tip of unit 110, and the display 152 is configured to display live direct visual images 156 from the camera module on the distal tip of unit 110. According to some embodiments, display monitors 150 and 152 can be touch sensitive for receiving user input as well as high resolution. According to some embodiments, each of the displays 150 and 152 are configured to display high definition graphics at pixel resolutions of 1280×720, 1920×1080, 2048×1080, 2560×1440, 3840×2160, or higher. According to some embodiments, the camera image display 152 shows live video image 156 in an orientation of anatomy inside of human cavity and relative position of anatomy with the tip of camera. The ultrasound image 154 shown on display 152 is generated by an ultrasound transducer at a known position relative to the camera. According to some embodiments, the ultrasound image and camera image can be precisely correlated in position and orientation inside the human cavity (e.g. uterus 102, shown in FIG. 1). The side by side display of anatomy ultrasound 154 and camera image 156 allows physicians to simultaneously see the ultrasound image guided by camera image in real time. According to some embodiments the camera module on the distal tip of unit 110 is configured and mounted such that at least a portion of the ultrasound probe head 252 is visible the operator in camera image 156 as ultrasound probe head portion 158. Providing ultrasound probe head portion 158 on live camera image 156 can provide valuable information and feedback to the operator as to the current orientation and position of ultrasound probe head 252.

According to some embodiments, processing system 170 includes ultrasound image processing unit 180, endoscopy image processing/hysteroscope unit 182 and image display and management system/graphics unit 184. The handheld unit 110 is connected to ultrasound processing unit 182 and hysteroscope unit 180 via cables 134 and 136, respectively. Processing system 170 can also include a suitable personal computer or a workstation that includes one or more processing units 174, input/output devices such as CD and/or DVD drives, internal storage 142 such as RAM, PROM, EPROM, and magnetic-type storage media, such as one or more hard disks 172 for storing the medical images and related databases and other information, as well as graphics processors suitable to power the graphics being displayed on displays 150 and 152. According to some embodiments, tower system 112 is powered by a medical grade power supply (not shown). According to some embodiments, a fluid control system 186 is attached to the handheld portion 110 via fluid line(s) 132. In some case there are two fluid lines so that in-flow and out-flow fluids can both be controlled.

FIG. 1B illustrates a system similar to FIG. 1A with a different display configuration, according to some embodiments. Instead of two displays, a single display monitor 150 can be used as shown in FIG. 1B. The endoscopy image 156 and ultrasound image 154 are combined by graphics unit 184. The endoscopy image 156 and ultrasound image 154 are displayed side by side simultaneously on a high-resolution single monitor 150 as shown. In some cases, this display configuration can give physicians a better visual effect during diagnostic and surgical procedures.

FIG. 1C shows another example of a combined ultrasound and endoscopy system (CUES), according to some embodiments. In this example, the CUES system 100 includes the handheld portion 110 being attached to two separate tower systems 116 and 118. Each of the systems 116 and 118 can be identical or similar to tower system 112 shown in FIGS. 1A and 1B. In the case shown in FIG. 1C, however, the ultrasound images 154 are being displayed on monitor 150 of tower system 116, while the endoscopy images 156 are being displayed on monitor 152 of tower system 118. The two tower systems 116 and 118 are positioned such that the monitors 150 and 152 are side by side, so that the physician can view both images simultaneously. Note that in this case, the hysteroscope unit 180 may be included in tower system 118 and the ultrasound processing unit 182 may be included in tower system 116.

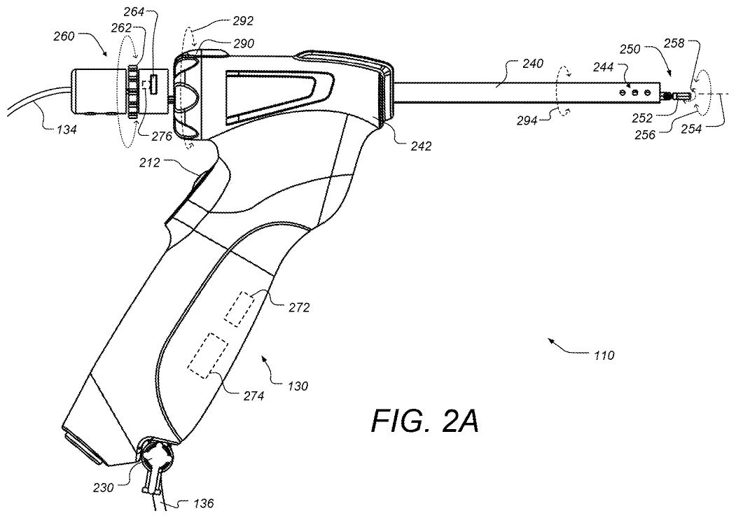

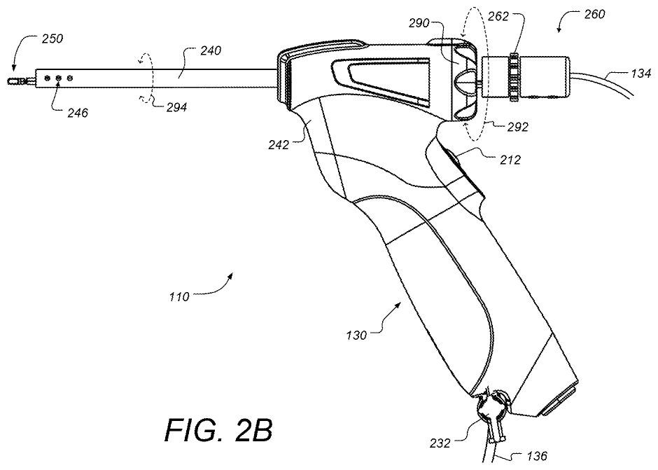

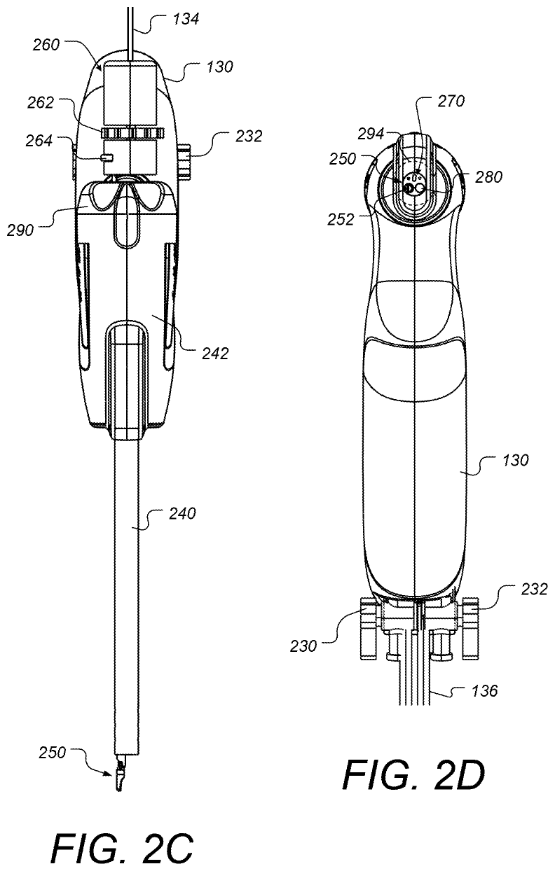

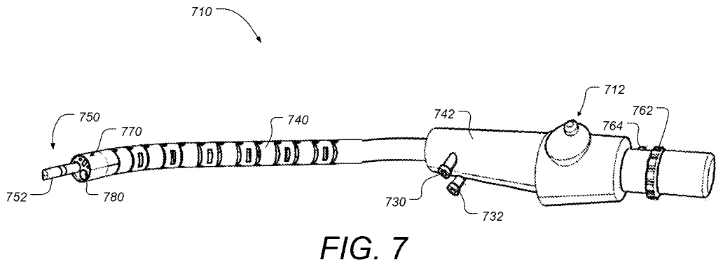

FIGS. 2A, 2B, 2C and 2D are right, left, top and front views of the handheld portion of a combined ultrasound and endoscopy system (CUES), according to some embodiments. The handheld portion 110 includes a cannula 240 and a distal tip 250. According to some embodiments, cannula 240 can be rigid, flexible (such cannula 740 in FIG. 7) or semi-rigid. In the case of semi-rigid, the cannula 240 can be made of a slightly bendable material such that the operator can slightly deflect the cannula, for example by 5-20 degrees, by manually pushing or pulling on the cannula shaft. The distal tip includes an ultrasound probe head 252 that can be rotated and “steered.” In particular, the ultrasound probe head 252 is configured to be rotated about a central longitudinal axis 254 as shown by dotted arrow 256, as well as steered as indicated by dotted arrow 258 to angle the ultrasound probe head 252 relative to axis 254. According to some embodiments, the rotation and steering of probe head 252 are both controlled at the proximal end 260 of cannula 240. According to some embodiments, at proximal end 260 the steering of probe head 252 is controlled by turning a wheel 262, and the rotation of probe head 252 is controlled by turning a wheel 264. According to some embodiments, mechanisms other than a wheel can be used to control the rotation of probe head 252. Such mechanisms include, without limitation, levers, sliders, sticks and trackballs.

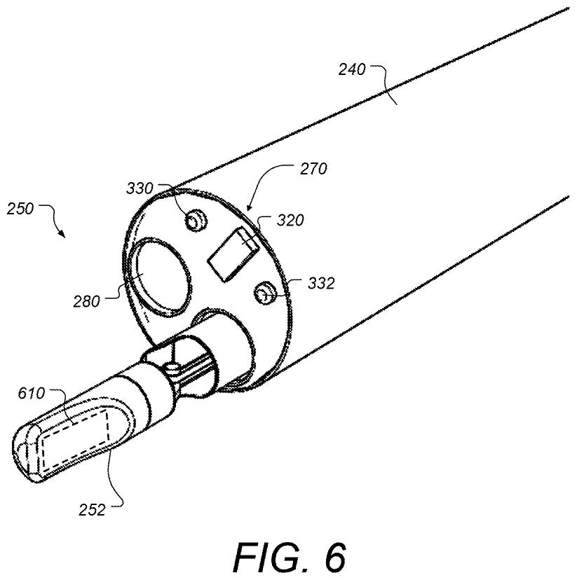

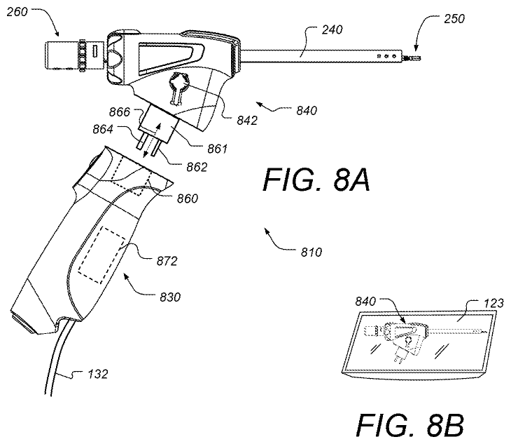

According to some embodiments, the ultrasonic probe head 252 includes an ultrasound transducer made from a traditional piezoelectric material such as PZT or from semiconductor materials. The dimension of the transducer can be 2-4 mm width and 10-20 mm length. The transducer in head 252 can include up to 128 elements (or more). See e.g. ultrasound transducer array 610 in FIG. 6. According to some embodiments, the ultrasound transducer array can be configured as a linear or phased array, or single element transducer having a frequency from 5 MHz to 10 MHz for general imaging to cover the entire uterus and bladder; and 10 MHz to 30 MHz for superficial uterus endometrial and bladder lining images. According to some embodiments, when fewer transducer elements are used in the array in head 252, for example, 64 elements or fewer, each transducer element can have its own cable. The cables can be bundled together and connected to an ultrasound processing unit (such as unit 182 shown in FIG. 1). According to some embodiments, ultrasound processing unit 182 can be integrated into and form part of processing system 170 (shown in FIG. 1), and in some other embodiments, some or all of ultrasound processing unit 182 can be separated from processing system 170. According to some embodiments, in the cases handle 130 is configured as part of the single-use portion, the handle 130 can include compact ultrasound processing unit 272 as shown in FIG. 2A. The compact ultrasound processing unit 272 in handle 130 can be made at lower cost and preform less functionality than cases where the handle is not configured to be single-use, such as handle 830 shown in FIG. 8A. According to some embodiments, in order to reduce the number of cables running from probe head 252, an ASIC can be included in probe head 252. The ASIC can include high voltage switches and control circuits to drive the individual transducer elements and route echo signals to processing unit 252. In such cases a reduced number of coaxial cables can then be used to carry ultrasound transmit and receive signals, control signals and electrical power between probe head 252 and ultrasound processing unit 182.

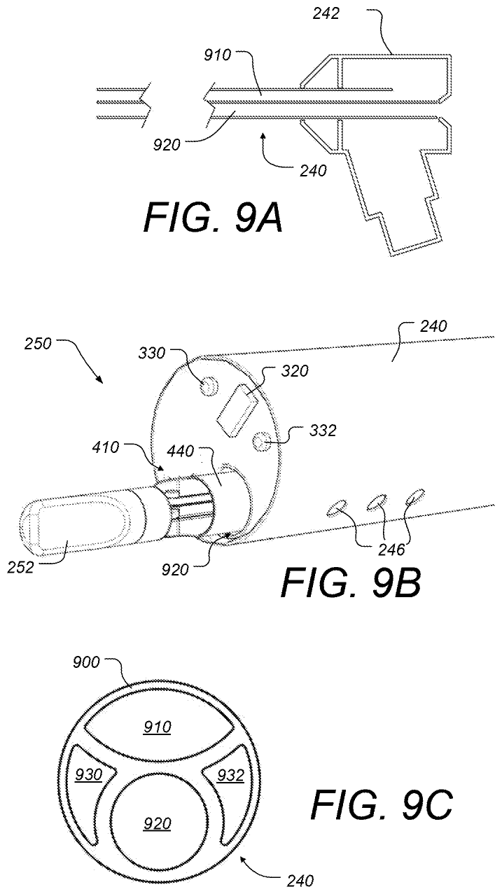

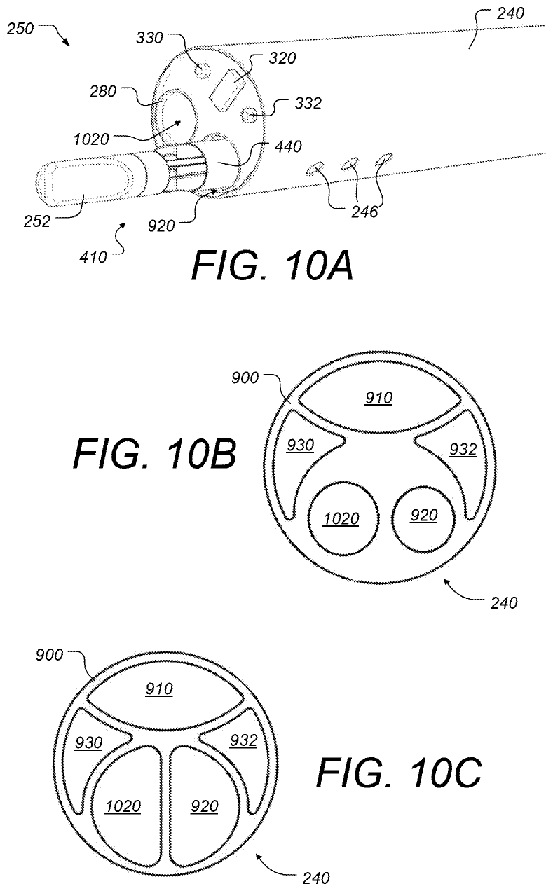

Referring again to FIGS. 2A-2D, the cannula 240 can be long, thin, and rigid or semi-rigid. According to some embodiments, the cross-section of cannula 240 perpendicular to its main longitudinal axis may be substantially circular. It should be noted the cross-section may have any suitable shape, such as oval. The diameter of the cannula may differ depending on the sort of endoscopy, such as from 3 mm and up to 15 mm. The cannula can include a working channel (working channel distal port 280 is shown in FIG. 2D). The working channel can be accessed from a port (not shown) on the rear end of proximal end 260. Cannula 240 may include one or more fluid channels in fluid communication with various fluid ports. The cannula 240 may include one channel to be shared by an inflow and an outflow. Alternatively, the cannula may include two or more channels with separate inflow and outflow. According to some embodiments, cannula 240 also includes one or more fluid lumens that are fluidically isolated from the working channel. The fluid lumens can lead to fluid ports 246 and 244 that are disposed on the left and right sides, respectively, of the distal end of the cannula 240. The fluid lumens can be in fluid communication with the fluid ports 230 and 232 at the bottom of the handle 130. According to some embodiments, the right-side fluid port 230 is connected to the right-side fluid ports 244, and the left-side fluid port 232 is connected to the left-side fluid ports 246. Cannula 240 is also configured to accommodate a plurality of electrical conductors used to provide power, control signals to and receive video/image data from to the camera and lighting modules 270 and ultrasound probe head 252 at distal tip 250. In some cases, the conductors can be insulated and disposed within a separate lumen within cannula 240, in other cases some or all of the conductors can be disposed within a lumen that is also used for another purpose (e.g. fluid and/or device/tool channel). According to some embodiments one or more optical fibers can pass through cannula 240 for purposes of data transmission and/or supplying illumination light to distal tip 250. Cannula 240 can also include one or more separate lumens for one or more of the rods or bars used to control the rotation and/or steering of the ultrasound probe head 252. Further details of possible cannula designs are shown in FIGS. 9A-9C and 10A-10C. Handle portion 130 includes a main body that is dimensioned and shaped to allow secure and ergonomic grasping by the operator's hand. Handle portion 130 also includes one or more buttons 212 that can be configured to allow execution of common tasks during use. For example, the button 212 can be programmed to control LED lighting level (of LEDs at the distal tip 250), capture still images and/or start and stop recording to video images, and capture and/or start and stop recording to ultrasound data and/or ultrasound images. According to some embodiments, an upper housing 242 can also be provided as shown.

According to some embodiments, the camera and lighting modules 270 at the distal tip of cannula 240 include a camera module that has a field of view of approximately 120 degrees. According to some embodiments, the camera module can be mounted such that its direction of view (DOV) is at an oblique angle from the main longitudinal axis of cannula 240. According some embodiments, the oblique angle is between zero and 30 degrees. An optical component, such as a prism can also be used to provide this oblique angle. Further details of techniques for altering the direction of view of the camera module can be found in co-pending U.S. patent application Ser. No. 16/268,819, which is incorporated herein by reference.

According to some embodiments the cannula 240 is connected to a rotation wheel 290 that is located near the proximal end of upper housing 242. By turning the cannula rotation wheel 290, as indicated by dashed arrow 292, the cannula 240, and the camera and lighting modules 270 can also be rotated, as indicated by dashed arrow 294. According to some embodiments, the cannula 240 can be configured to rotate by 180 degrees (or more), such that camera and lighting modules 270 can provide at least of 180 degrees viewing angle in the uterus, bladder and other organ cavities.

According to some embodiments, the handle portion 130 contains a set of electronics 274 which filter and transmit raw image data to hysteroscope image processing unit 180 that can be part of processing system 170 (shown in FIG. 1). According to some embodiments, a rotation sensor 276 is included on wheel 264 and is configured to measure or detect the rotational position of the ultrasound probe head 252. The rotation sensor 276 data are used by the ultrasound processing unit 182 (shown in FIG. 1) for constructing three-dimensional ultrasound images.

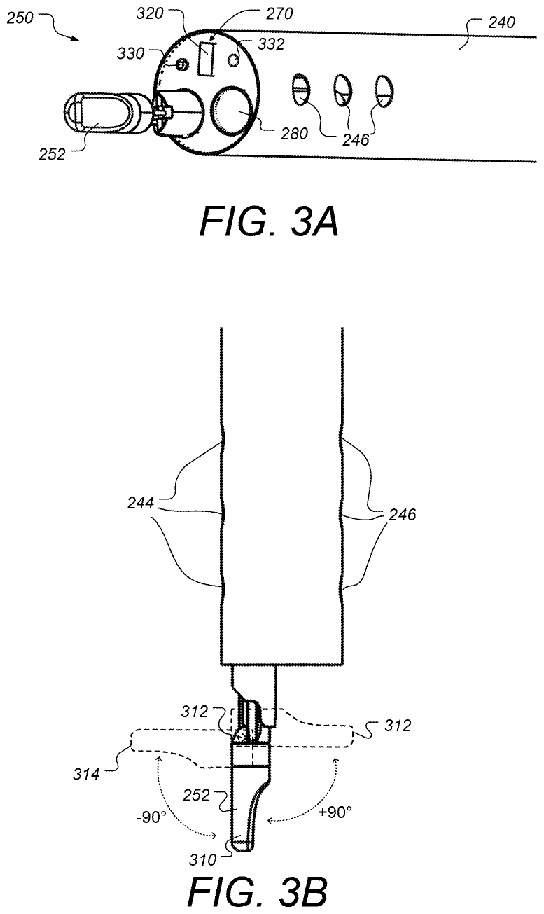

FIGS. 3A and 3B are perspective and top views of the distal end of a handheld portion of a combined ultrasound and endoscopy system (CUES), according to some embodiments. In FIG. 3A, the camera and lighting modules 270 are shown to include a camera module 320 and two LEDs 330 and 332. In FIG. 3B, the dotted outlines 312 and 314 illustrate the steering range of the ultrasonic probe 252. The probe 252 is shown in solid outline in a vertical or “neutral” position 310. In the example shown, the probe head 252 pivots about a hub 312 and can be positioned approximately 90 degrees in either direction, as shown. According to some embodiments, fluid irrigation can be provided by flowing fluid into and/or out of the working channel distal port 280 and/or the side fluid ports 246 and 244. In one case, the working channel distal port 280 is used for “in flow” or flowing fluid into the organ/tissues of interest while the side fluid ports 246 and 244 are used for “out flow” or receiving flowing fluid from the organ/tissues of interest. In this case, one of the fluid ports 230 and 232 at the bottom of the handle 130 can be used for in flow and the other can be used for out flow.

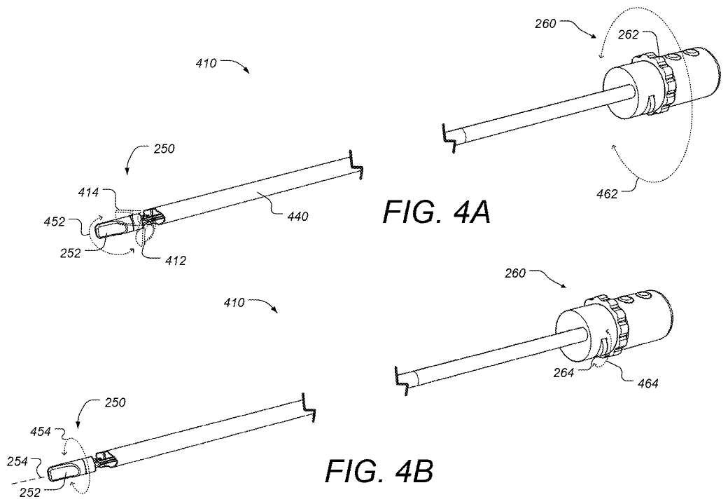

FIGS. 4A and 4B are partial perspective views of an ultrasound probe forming part of a combined ultrasound and endoscopy system (CUES), according to some embodiments. Shown is ultrasound probe assembly 410 that is configured for deployment through a channel of an endoscopic cannula. FIG. 4A illustrates further details of the steering control of the ultrasonic probe head 252. The rotation of wheel 262 at the proximal end 260, as indicated by dotted arrow 462, controls the left and right steering of probe head 252, as indicated by dotted arrow 452 and dotted outlines 412 and 414. According to some embodiments, mechanisms other than a wheel can be used to control the steering of probe head 252. Such mechanisms include, without limitation, levers, sliders, sticks and trackballs. Also shown is shaft 440 that runs the length of the assembly 410 from the probe head 252 to the proximal end 260. FIG. 4B illustrates further details of the rotation control of the ultrasonic probe head 252 about axis 254, as indicated by arrow 454. The rotation of the probe head 252 is controlled by rotating wheel 264, as indicated by arrow 464. According to some embodiments, the entire ultrasound assembly 410 shown in FIGS. 4A and 4B can be configured for use in the working channel of a conventional endoscopy systems to provide ultrasonic capability to an endoscopy system, thereby forming a combined ultrasound and endoscopy system (CUES). In such cases, a cable connector (not shown) is provided at the proximal end 260 of the assembly 410. The cable connector is used for power, control, and transmitting and receiving ultrasound signals and/or data from and to the ultrasound probe head 252.

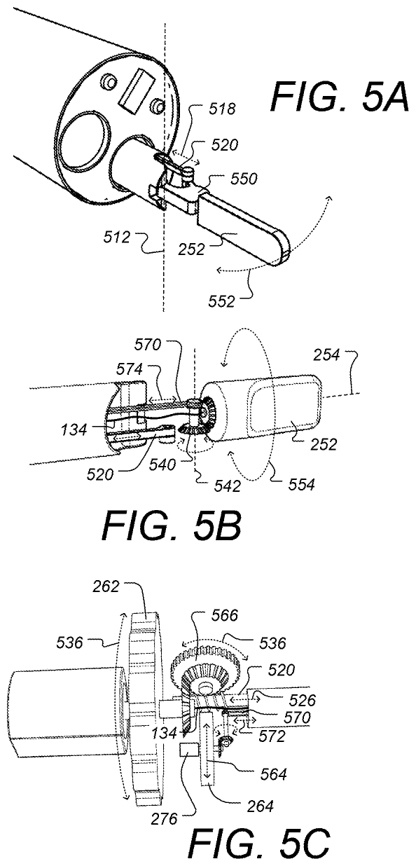

FIGS. 5A, 5B and 5C are perspective views showing further details of mechanisms for steering and rotation of an ultrasonic probe head that forms part of a combined ultrasound and endoscopy system (CUES), according to some embodiments. FIG. 5A shows further detail of the steering of the ultrasonic probe head 252. The steering motion is indicated by the dotted arrow 552. According to some embodiments, the steering is controlled by the axial translation of a push bar 520, as indicated by arrow 518. Moving bar 520 forward or backwards pushes or pulls the rotation plate 550 which pivots on a hub (not shown) about axis 512. According to some embodiments, rotation plate 550 is configured to provide a range of motion of at least 120 degrees. In some embodiments, the steering system can be configured to provide equal amounts of steering range in either direction, but in other embodiments the steering range is not equal, which provides greater steering/probe deflection in one direction. Note that by providing rotation of the probe head 252 and/or rotation of the entire cannula shaft 240, the ultrasonic transducers array 610 (shown in FIG. 6) in probe head 252 can be oriented with respect to the tissues of interest over a relatively wide range, thereby enhancing the ability for clear and useful ultrasonic imaging.

FIG. 5B shows further detail of the rotation of the ultrasonic probe head 252. The rotational motion about axis 254 is controlled by moving a belt 570, as indicated by arrow 574. The belt 570 rotates a gear piece 540 about axis 542. Gear piece 540 has a bevel gear that meshes with a bevel gear on the proximal end of probe head 252. Also visible in FIG. 5B is steering control push bar 520. According to some embodiments, the probe head 252 can be configured with a range of rotational motion about axis 254 of approximately 360 degrees.

FIG. 5C illustrates further detail of the operation of the distal end steering and rotational control wheels 262 and 264. Steering control wheel 262 rotates as indicated by arrow 536. The wheel 262 connected via a rod to a bevel gear that meshes with a bevel gear on wheel 566 which rotates as indicated by arrow 536. The wheel 566 has another gear surface that meshes with a worm gear formed on steering control push bar 520. The imparted axial motion of bar 520 is indicated by arrow 526. Also shown in FIG. 5C is rotation control wheel 264 which rotates as indicated by arrow 564. The wheel 264 turns a bevel gear that meshes with another bevel gear which in turn moves belt 570, as indicated by arrow 572. Rotation sensor 276 is also shown. The rotation sensor 276 can be connected to the hub of wheel 264 as shown or it can be positioned inside of rotation wheel 264. The rotation sensor 276 can be an optical encoder or another meter such as potentiometer. The rotation sensor 276 provides the ultrasound transducer position where a frame of ultrasound data is acquired. The position information is attached to a frame of ultrasound data which will be used to construct a 3D ultrasound image. The rotation position information can also be sent to the ultrasound processing unit 182. According to some embodiments, the position information can be used to trigger an ultrasound timing control unit to start transmitting ultrasound waves and receiving ultrasound data. Using the rotation position sensing from sensor 276, the distance of two frames of ultrasound images can be precisely controlled to about 0.1 mm spatial resolution.

FIG. 6 is a perspective view illustrating further detail of the distal tip of a combined ultrasound and endoscopy system (CUES), according to some embodiments. The distal tip 250 includes the camera and lighting modules 270, the working channel distal port 280 and ultrasound probe head 252. According to some embodiments, the working channel has an inner diameter of about 3.2 mm such that standard surgical devices can be disposed therein to carry out various surgical procedures. Examples of such devices include: biopsy needles, injection needles, forceps, tubes, knives, snares, probes, coagulator devices, brushes, laser devices, microwave devices (e.g. for ablation), and photodynamic tools. The camera and lighting modules 270 include camera module 320 and two LEDs 330 and 332. According to some embodiments, a camera module 320 can include a CMOS image sensor such as model number OVM6946 from OmniVision Technologies, Inc. According some embodiments, camera module 320 is about 1.05 mm×1.05 mm, has about 400×400 pixel resolution, and has about a 120-degree field of view, and can capture video at 30 frames per second.

FIG. 7 is a perspective view of a flexible handheld portion forming part of a combined ultrasound and endoscopy system (CUES), according to some embodiments. The flexible handheld portion 710 is configured to be connected to a tower system such as by cables 134 and 136 to a tower system 112, as shown in FIG. 1. Referring again to FIG. 7, the cannula 740 has flexible cables that provide for steering or bending of the cannula 740 in multiple directions. According to some embodiments, both rotation and steering mechanical control can be provided, for example as illustrated in FIGS. 3B and 5A-C2A. Handheld portion 710 includes a proximal handle portion 734 configured for ergonomic grasping by hand According to some embodiments, the flexible handheld portion 710 includes a distal tip 750 that includes a camera and lighting modules 770, ultrasonic probe head 752, and working channel distal port 780. Those components can be similar or identical to camera and lighting modules 270, ultrasonic probe head 252, and working channel distal port 280 described and shown elsewhere herein. In cases where handheld portion 710 is integrated and has mechanical rotation and steering controls, the ultrasound probe head 752 need not include its own rotation capability. According to some embodiments, the ultrasound probe head 752 is configured to reduce steering and/or rotation range of motion when compared to probe head 252, shown and described elsewhere herein. According to some embodiments, two proximal fluid ports 730 and 732 are configured to provide fluid in flow and fluid out flow and are analogous to fluid ports 230 and 232 shown in FIGS. 2A-2D.

According to some embodiments, a flexible CUES such as shown in FIG. 7 can be more suitable for male urological patients than a rigid CUES. For example, locations in the bladder can be more easily accessed and less painful for male patients when using a flexible CUES. According to some embodiments, the flexible CUES can be used to better access the ureter and make ultrasound scans of the kidney. The flexible CUES may also be better suited to reach and obtain ultrasound images of certain locations in the uterus that a rigid hysteroscope cannot easily access.

FIGS. 8A and 8B are diagrams showing further detail of separable handheld portion of a combined ultrasound and endoscopy system (CUES), according to some embodiments. In this case the handheld portion 810 has many components that are similar and/or identical to handheld portion 110 shown and described elsewhere herein. In this case, handheld portion 810 is configured to be separable into an upper single-use portion 840 and a lower multiple-use portion 830. The two portions 830 and 840 are able to mate and un-mate with each other by hand-operation that does not need tools. In particular, handle portion 830 includes a socket 860 that is dimensioned to couple with male mating portion 861 that protrudes from single-use portion 840. The action of mounting and un-mounting is shown by dotted arrow 866. Protruding from mating portion 861 are electrical connectors 862 and 864. According to some embodiments, the handle portion 830 may house or comprise components configured to process image data, generate control signals, provide power, or establish communication with other external devices. In some cases, the communication may be wireless or wired communication. For example, the wireless communications may include Wi-Fi, radio communications, Bluetooth, IR communications, or other types of direct communications. In some embodiments, the handle portion may house a sensor assembly to measure a relative position between the cannula and the handle portion. In other embodiments, the sensor assembly may measure relative position or orientation of the handle to its environment. Examples of such sensor assemblies are described further below. FIG. 8B shows the single use portion 840 being supplied in a sterile package or pouch 123. Since the lower portion of the handle 830 is configured for multiple-use (rather than single-use, such as handle 130 shown in FIGS. 2A-2D), according to some embodiments greater processing capabilities are incorporated therein. For example, electronics unit 872 can be included in lower handle portion 830 to provide some or all of the functionality of ultrasound processing unit 182 (shown in FIG. 1).

Further detail of the operation of a combined ultrasound and endoscopy system (CUES) will now be provided. In the case of a rigid endoscope and ultrasound probe, such as handheld portions 110 and 810 shown and described herein, the following sequence may be used: (1) Under the guidance of live video images from the camera module 320 (shown in FIGS. 3A and 6) on the endoscope tip, the scope is inserted into uterus or bladder. (2) For imaging the uterus cervical canal, the ultrasound probe is oriented in its vertical position (e.g. position 310 in FIG. 3B). Rotation control wheel 264 is manipulated to rotate ultrasound probe 252 about axis 254 (e.g. shown in FIG. 5B). Ultrasound images of the cervix wall and/or vertical section part of uterus wall are thereby obtained. (3) The distal tip 250 is moved into the uterus or bladder cavity. The steering control wheel 262 is manipulated to adjust probe head 252 to a desired angle, then the rotation control wheel 264 is manipulated to rotate ultrasound probe head 252 about axis 254 (e.g. shown in FIG. 5B), thereby obtaining ultrasound images of the upper uterus or upper bladder wall(s).

In the case of a flexible endoscope and ultrasound probe, such as handheld portions 710 shown in FIG. 7, the following sequence may be used. (1) The distal tip 750 of flexible cannula 740 is inserted into the uterus or bladder cavity. During insertion, the ultrasound probe head 752 can be in a retracted position, where the distal tip of the probe head 752 is recessed proximally from the distal end of cannula 740. (2) Live video endoscopy images from the camera module are used to find an area where the ultrasound image should be taken. (3) The ultrasound probe is pushed distally such that probe head 752 protrudes distally from the distal tip 750 (such as shown in FIG. 7). (4) The ultrasound steering control wheel 762 is used to adjust probe head to a desired angle, then ultrasound rotation control wheel 764 is manipulated so as to rotate ultrasound probe head 752 to a desired angle, thereby obtaining suitable ultrasound images.

FIGS. 9A-9C are diagrams illustrating further detail of the distal tip and cannula of a combined ultrasound and endoscopy probe, according to some embodiments. FIG. 9A is a schematic cross-section diagram that shows the cannula 240 including an upper lumen or channel 910 and a lower lumen or channel 920. FIG. 9B is a perspective view of the distal tip 250 showing ultrasound probe assembly 410 deployed in the lower channel 920. Note that in the example shown in FIGS. 9A-9C, the ultrasound probe assembly 410 is being deployed in cannula 240 that is configured with a single working channel (channel 920). In some cases, the cannula 240 can include multiple working channels, such as shown in FIGS. 10A-10C. FIG. 9C shows a cross section of the cannula 240 having a single working channel. In this case cannula 240 has an outer wall 900 that houses four separate channels. The upper channel 910 is used for cables to and from the camera module 320, LEDs 330 and 332 and possibly other electrical and/or fiber optic cable(s). The two side channels 930 and 932 are used for out-flow liquid (i.e. flowing liquid from the patient's organ or cavity out into the device). The side channels 930 and 932 can be fluidly connected to the side fluid ports 244 (shown in FIGS. 2A and 3B) and 246, respectively. The lower channel 920 is shared by in-flow liquid (i.e. flowing liquid into the organ or cavity), ultrasound probe 410 and possibly other surgical tools. According to some embodiments, lower or working channel 920 has an inner diameter ranging from 1 mm to 4 mm. According to some embodiments, the overall outer diameter of endoscope cannula 240 in the cases of a single working channel, as shown in FIGS. 9A-9C, is less than about 7 mm. In a surgical operation, the uterus or bladder cavity is filled with liquid through channel 920. A surgical tool can be inserted into the cavity to perform surgical procedure. During the surgical operation, the surgeon can withdraw the surgical tool and insert ultrasound probe 410 through working channel 920 to measure cavity wall thickness, the shape of a surgical objective and obtain other information so that surgeon can make decision for completion of the surgical procedure.

FIGS. 10A-10C are diagrams illustrating further detail of the distal tip and cannula of a combined ultrasound and endoscopy probe, according to some embodiments. In the cases shown, the cannula 240 includes two working channels. FIG. 10A is a perspective view of the distal tip 250 in the case where cannula 240 includes two working channels 920 and 1020. In the case shown, ultrasound probe assembly 410 is deployed through working channel 920 while working channel 1020 is empty. FIG. 10B is a cross-section of cannula 240. As in the case of the single working channel, there is an upper channel 910 that is used for cables for the camera module 320, LEDs 330 and 332 and possibly other electrical and/or fiber optic cable(s). Similarly, the two side channels 930 and 932 are used for out-flow liquid (i.e. flowing liquid from the patient's organ or cavity out into the device) through the side fluid ports 244 (shown in FIGS. 2A and 3B) and 246, respectively. The working channel 920 is used for the ultrasound probe assembly 410 while a separate working channel 1020 can be used for both liquid in-flow, and surgical tool(s). According to some embodiments, the diameter of each of the working channels 920 and 1020 range from 1 mm to 4 mm, and the overall outer diameter of endoscope cannula 240 is less than about 10 mm. In a surgical operation, the uterus or bladder cavity is filled with liquid through channel 1020 and the ultrasound probe 410 is inserted into the uterus or bladder cavity through channel 920. After the uterus or bladder cavity is appropriately filled with liquid (e.g. saline), the surgical tool is inserted into the uterus or bladder cavity through channel 1020. During the surgical operation, the use of the surgical tool can temporarily be stopped while the ultrasound probe is moved to the surgical operation area to generate ultrasound image. The ultrasound image(s) can provide guidance about uterus or bladder cavity wall thicknesses, on the shape of a surgical objective, and other information useful for making surgical decisions. If a biopsy operation is being performed, the ultrasound image can guide a biopsy tool to collect tissue samples.

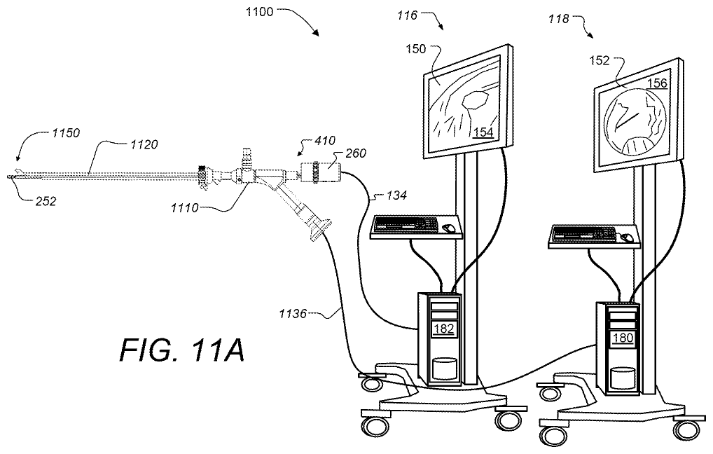

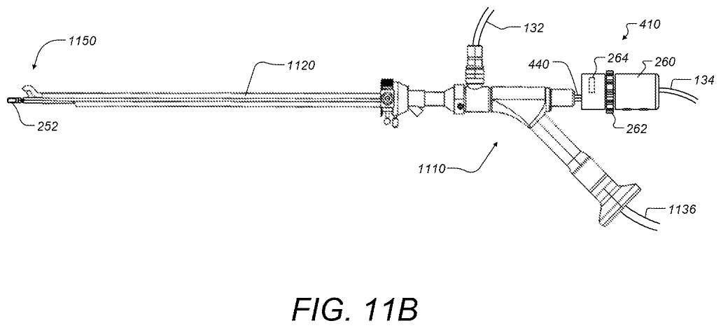

FIGS. 11A and 11B are schematic diagrams showing further examples of combined ultrasound and endoscopy systems (CUES), according to some embodiments. In these examples, ultrasound probe assembly 410 is inserted into the working channel of a conventional reusable rigid, semirigid or flexible hysteroscopes, cystoscope, or other conventional endoscopy system to form a combined ultrasound and endoscopy systems (CUES). The ultrasound probe assembly 410 can be similar or identical to the ultrasound probe assemblies described elsewhere herein including controllable steering and rotating capabilities of the probe head 252. In FIG. 11A, CUES system 1100 is formed by inserting ultrasound probe assembly 410 through the working channel of rigid hysteroscope 1110. As can be seen in FIGS. 11A and 11B, the shaft 440 of ultrasound probe assembly 410 is inserted into the working channel of cannula 1120 such that the ultrasound probe head 252 protrudes from the distal tip 1150 of hysteroscope 1110. Hysteroscope 1110 includes a proximal handle portion 1130 configured for ergonomic grasping by hand. In FIG. 11A, the CUES system 1100 includes two tower systems 116 and 118 similar or identical to those shown in FIG. 1C. In the case of FIG. 11A, hysteroscope 1110 is attached via cable 1136 to hysteroscope unit 180 of endoscopy tower system 118. Endoscopy tower system 118 includes monitor 152 that is configured to display hysteroscopy images 156 as shown. Not shown are fluid lines which run to fluid control system. The conventional hysteroscope 1110 and tower system 118 can be a conventional, stand-alone endoscopy system. The ultrasound assembly 410 is connected via cable 134 to ultrasound processing unit 182 which in this case is located in ultrasound tower system 116. Ultrasound tower system 116 includes monitor 150 that is configured to display hysteroscopy images 154 as shown. In this way, the ultrasound probe assembly 410, cable 134 and tower system 116 form a stand-alone ultrasound unit. By positioning the tower systems 116 and 118 close to each other so that monitors 150 and 152 can be viewed by the operator(s) simultaneously, the CUES system 1110 can provide benefits as described elsewhere herein including: allowing the operator(s) to see both the surface and inside tissue of organs; and providing an ultrasound image guided by a camera image in real time thereby enhancing the effectiveness of various diagnostic and surgical procedures.

Examples of procedures that can be performed using the combined ultrasound and endoscopy systems (CUES) described herein include, without limitation: endometrial cancer detection, screening, and/or diagnosis; ultrasound-based surgical planning; ultrasound-based therapy planning; surgical monitoring; and uterine surface roughness assessment. According to some embodiments, the combined ultrasound and endoscopy systems (CUES) described herein can be used for monitoring gynecology and urology surgeries such as: uterine wall resection; endometrial ablation; endometrial resection; submucous myoma resection; intramural myoma resection; transmural myoma resection; resection of cervix and/or cervical canal; prostate resection; and uterine myomectomy. The combined ultrasound and endoscopy systems (CUES) described herein can also be used for making measurements such as: uterine wall thickness; endometrium thickness; polyp size; prostate thickness; intra uterine measurements; urethra thickness. The combined ultrasound and endoscopy systems (CUES) described herein can also be used to generate three-dimensional images of various organs and body parts, such as: ovaries; fallopian tube(s); uterus; prostate; and various tumors and/or polyps.

Although the treatment planning and definition of treatment profiles and volumes described herein are presented in the context of urological or gynecological diagnosis or surgery, the methods and apparatus as described herein can be used to treat any tissue of the body and any organ and vessel of the body such as brain, heart, lungs, intestines, eyes, skin, kidney, liver, pancreas, stomach, uterus, ovaries, testicles, bladder, ear, nose, mouth, soft tissues such as bone marrow, adipose tissue, muscle, glandular and mucosal tissue, spinal and nerve tissue, cartilage, hard biological tissues such as teeth, bone and the like, as well as body lumens and passages such as the sinuses, ureter, colon, esophagus, lung passages, blood vessels and throat.

The embodiments disclosed herein can be combined in one or more of many ways to provide improved diagnosis and therapy to a patient. The disclosed embodiments can be combined with prior methods and apparatus to provide improved treatment, such as combination with known methods of urological, or gynecological diagnosis, surgery and surgery of other tissues and organs, for example. It is to be understood that any one or more of the structures and steps as described herein can be combined with any one or more additional structures and steps of the methods and apparatus as described herein, the drawings and supporting text provide descriptions in accordance with embodiments.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An integrated visual and ultrasound device comprising:
an ergonomic handle (130) configured for grasping by hand and having proximal and distal portions;
a cannula (240) extending distally from the distal portion of the handle and having a distal portion extending along a longitudinal axis (254);
a distally facing camera (320) secured at the distal portion of the cannula and having a camera field of view (FOV) encompassing a selected solid angle and a camera direction of view (DOV);
an ultrasound probe (252) positioned at the distal portion of the cannula for both rotation about said axis relative to the distal portion of the cannula and for tilting relative to said distal portion of the cannula;
a probe steering mechanism (534) mounted at the proximal end of the handle and operatively coupled with the ultrasound probe to selectively tilt the ultrasound probe relative to said distal portion of the cannula over a selected angular range; and
a probe rotation mechanism (536) mounted at the proximal end of the handle and operatively coupled with the ultrasound probe to selectively rotate the ultrasound probe about said axis relative to the cannula.

2. The device of claim 1, in which said cannula further comprises at least one lumen (910, 920, 1020) and further including a shaft (440) connecting the probe steering mechanism and the ultrasound probe and configured to tilt the probe relative to said distal portion of the cannula, wherein said shaft is received in said lumen.

3. The device of claim 2, further including a belt (570) that is in said shaft is coupled to and driven by said probe rotation mechanism and further including gearing (540) secured to said ultrasound probe and driven by said belt to selectively rotate the ultrasound probe about said longitudinal axis.

4. The device of claim 1, in which said probe rotation mechanism is configured to rotate the ultrasound probe through at least 180 degrees about said longitudinal axis.

5. The device of claim 1, further including a rotation plate (550) to which the ultrasound probe is secured and which rotates about a pivot axis (542) transverse to said longitudinal axis, and a bar (520) inside said shaft, said bar coupling said probe steering mechanism to said rotation plate and responding to manual actuation of the steering mechanism to pivot the rotation plate and thus the ultrasound probe relative to said distal portion of the cannula.

6. The device of claim 1, in which said steering mechanism is configured to selectively tilt said ultrasound probe in two opposite directions (312, 314) relative to said distal portion of the cannula through an angle up to 180 degrees in at least one of said directions.

7. The device of claim 6, in which said steering mechanism is configured to tilt said ultrasound probe through different angular ranges in said two opposite directions relative to said distal portion of the cannula.

8. The device of claim 1, in which said handle comprises (i) a multiple-use portion (130) and image processing electronics therein (272, 274) coupled to said camera and said ultrasound probe and (ii) a single-use portion (120) removably secured to the multiple-use portion and housing said rotation mechanism and steering mechanism.

9. The device of claim 1, in which said cannula is flexible (740) and bends when inserted into a patient's bladder or ureter.

10. The device of claim 1, further including an ultrasound image processor (182) operatively coupled with said ultrasound probe and an ultrasound image display (150) configured to display ultrasound images (154) provided by said ultrasound probe and processed by said ultrasound processor, and a camera image processor (180) and a camera image display (152) configured to display images provided by said camera (156, 158) and processed by said camera image processor.

11. The device of claim 10, in which said ultrasound image display and camera image display are configured to concurrently display said ultrasound images (150) and camera images (156, 158), including selectively displaying concurrently a camera image (158) of the ultrasound probe.

12. The device of claim 1, wherein the ultrasound and visual aspects are integrated by the ultrasound probe being inserted through a working channel formed within the cannula.

13. The device of claim 1, wherein said cannula comprises a set of three cannulas (121, 122, 123) one of which is rigid, another semi-rigid and yet another flexible, wherein only a selected one of said three cannulas is secured to said handle at any one time.

14. The device of claim 1, further comprising a cannula rotation mechanism (290) positioned at the proximal portion of the handle and operatively coupled with the cannula to selectively rotate the cannula and thus the camera about said axis relative to the handle.

15. The device of claim 14 in which the probe rotation mechanism is configured to rotate the probe about said longitudinal axis relative to the cannula.

16. The device of claim 1, wherein said probe rotation mechanism is a probe rotation wheel (264).

17. The device of claim 1, further comprising a rotation sensor (276) operatively coupled with the probe rotation mechanism and configured to provide an electronic signal indicative of rotation of the ultrasound probe about said longitudinal axis.

18. The device of claim 1, in which said camera direction of view (DOV) is in the range of greater 0 to 30 degrees.

19. A medical device comprising:
an elongated shaft (440) having a distal portion (250) extending along a longitudinal axis (254) and a proximal portion (260), wherein the shaft is shaped and dimensioned for insertion into a sheath or a working channel (920) of an endoscope cannula configured for insertion into a patient;
an ultrasound probe (252) that is at the distal portion of the shaft and is configured to protrude from a distal end of the sheath or cannula and to provide ultrasound images;
a housing secured to the proximal portion of the shaft;
a probe rotation mechanism (264) mounted on or in the housing and operatively coupled with the shaft to rotate the shaft and thus the ultrasound probe about said axis over a selected rotation angular range; and
a probe steering mechanism (536) mounted on or in the housing and operatively coupled with the ultrasound probe to tilt the ultrasound probe relative to a distal portion of said cannula over a selected tilting angle range.

20. The medical device of claim 19, further including a rotation sensor (276) operatively coupled with the probe rotation mechanism and configured to provide an electronic signal indicative of rotation of the ultrasound probe about said axis.

21. The medical device of claim 19, in which said shaft is shaped and dimensioned for insertion into a working channel of an endoscope that has a camera (320) at a distal end thereof, wherein the ultrasound probe is configured to protrude distally from said camera when the shaft in inserted into said working channel.

22. A medical device comprising:
a handle (734);
a flexible cannula (740) that extends distally from the handle and is configured to bend to a desired shape;
a distally facing camera (770) secured at the distal portion of the cannula and having a camera field of view (FOV) encompassing a selected solid angle and a camera direction of view (DOV);
an ultrasound probe (752) positioned at a distal portion of the cannula for both rotation about an axis extending along said distal portion of the cannula and for tilting relative to said distal portion of the cannula;
an ultrasound probe steering mechanism (762) mounted at a proximal portion of the handle and operatively coupled with the ultrasound probe to selectively tilt the ultrasound probe relative to said distal portion of the cannula over a selected angular range; and
an ultrasound probe rotation mechanism (764) mounted at the proximal portion of the handle and operatively coupled with the ultrasound probe to selectively rotate the ultrasound probe about said axis extending along said distal portion of the cannula through a selected rotation angle.

23. The medical device of claim 22, in which the angular range of said tilting of the ultrasound probe relative to said distal portion of the cannula is 0-30 degrees and the rotation angle of the ultrasound probe is at least 180 degrees in each direction.
